(19) United States
(12) Patent Application Publication
(10) Pub. No.: US 2026/0234571 A1
(43) Pub. Date: Aug. 13, 2026

(54) A CHROMATOGRAPHY SYSTEM, USE THEREOF, AND A METHOD FOR SEPARATING ADENO-ASSOCIATED CAPSIDS

(71) Applicant: Cytiva BioProcess R&D AB, Uppsala (SE)

(72) Inventors: Jean-Luc Maloisel, Uppsala (SE); Åsa Hagner McWhirter, Uppsala (SE); Ola Lind, Uppsala (SE); Brigitta Németh, Uppsala (SE)

(21) Appl. No.: 19/474,119

(22) PCT Filed: Apr. 8, 2024
(86) PCT No.: PCT/EP2024/059429

§ 371 Date: Oct. 9, 2025

(30) Foreign Application Priority Data
Apr. 12, 2023 (SE) 2350425-1

(51) Int. Cl. C12N 7/00; B01D 15/18; B01D 15/20; B01D 15/34; B01D 15/36; B01D 15/38; B01J 41/05; B01J 41/20; C12N 15/86

(52) U.S. Cl. CPC C12N 7/00; B01D 15/1871; B01D 15/203; B01D 15/34; B01D 15/362; B01D 15/363; B01D 15/3804; B01D 15/3847; B01J 41/05; B01J 41/20; C12N 15/86; C12N 2750/14151

(57) ABSTRACT

The present disclosure is directed to a chromatography system comprising a buffer valve arrangement configured to allow independent control of a first buffer feed and a second buffer feed; a pump arrangement configured to supply a first buffer feed, a second buffer feed, and a feed comprising a biological target compound and one or more impurities; a selection valve arrangement, comprising a first chromatography device selection valve; a first chromatography device comprising a first chromatography material comprising a support material functionalised with a ligand, wherein the ligand comprises an affinity group having a binding affinity for a biological target compound, an ion exchange group, or a multimodal group; a second chromatography device, comprising a second chromatography material, which comprises a conditioning chromatography material; a third chromatography device comprising a third chromatography material; wherein the selection valve arrangement is configured to enable separation of biological target compounds from impurities by allowing a feed, comprising biological target molecules and one or more impurities, to continuously pass through the first, the second, and the third chromatography devices, wherein the first, the second, and the third chromatography devices are configured to be connected in series. Further provided are a use of the chromatography system and a method for separation of adeno-associated virus capsids from one or more impurities.

### FIELD OF DISCLOSURE

The present disclosure relates to the field of separation of biological target compounds, such as adeno-associated capsids. The disclosure is directed to a chromatography system and use thereof for separating adeno-associated virus capsids from impurities, as well as a method for separating adeno-associated virus capsids from impurities.

### BACKGROUND OF THE DISCLOSURE

Liquid chromatography is a separation technique that is used to separate and analyse complex liquid mixtures of compounds. The process involves a stationary phase and a liquid mobile phase, where the liquid mixture to be separated is introduced to the mobile phase and passes through the stationary phase. The different components of the mixture will interact with the stationary phase to varying degrees, causing them to separate and be collected at different times. There are various types of liquid chromatography techniques that differ based on the stationary and mobile phases used, for example ion exchange chromatography, size-exclusion chromatography, and affinity chromatography. Different types of chromatography require different ways of sample preparation, different process conditions and buffers.

Various biological target compounds, such as antibodies and viral vectors, are produced by fermentation of host cells. The biological target compounds must then be separated from host cell material and other impurities contained in the fermentation broth before they can be used for example in medical and analytical applications. A combination of several steps of different types of liquid chromatography is often applied with the object of achieving a high recovery of the biological target molecule at a high level of purity. However, processes for separation of biological target compounds often require manually switching between different chromatography devices and sample preparation between chromatography steps to condition the sample for the next step, which altogether makes the processes time-consuming.

Adeno-associated viruses (AAV) are non-enveloped viruses that have linear single-stranded DNA (ssDNA) genome and that can be engineered to deliver DNA to target cells. Recombinant adeno-associated virus (rAAV) vectors have emerged as one of the most versatile and successful gene therapy delivery vehicles. To use AAV particles as vectors in therapy it is necessary to purify the virus particles from cell impurities like DNA after transfection. Further, since therapeutic efficacy of AAV vectors is dependent on high percentage of virus particles fully packaged with genetic material of interest, it is important to separate such fully packaged AAV from empty and partially packaged AAV particles. WO2023285011 A1 describes a method for separating fully packaged AAV particles from not fully packaged AAV particles. However, there is a continuous need in the art for novel chromatography systems providing higher capacities and faster processing, leading to a generally better process economy.

### SUMMARY OF THE INVENTION

One object of the present disclosure is to provide a chromatography system enabling a faster and simplified separation of biological target compounds from impurities, with similar performance as previously known chromatography systems. The chromatography system is constructed such that a feed, comprising biological target compounds and impurities, can be subjected to several purification steps uninterruptedly thanks to being continuously passed through the system. Several chromatography devices are connected in-line, within the chromatography system. Thereby, a significantly reduced time is achieved compared to when using conventional chromatography systems. Traditionally, purification process flows are typically interrupted and prolonged for example due to manually changing of buffers and/or chromatography devices within the system, and since some steps are performed in separate containers or devices, outside of the chromatography system, e.g., conditioning of chromatography media and/or performing of certain filtration/purification steps. With the presently disclosed chromatography system, less human intervention is required compared to when operating previously known chromatography systems.

More particularly, the present disclosure is directed to a chromatography system comprising: a buffer valve arrangement configured to allow independent control of a first buffer feed and a second buffer feed;
a pump arrangement configured to supply a first buffer feed, a second buffer feed, and a feed comprising a biological target compound and one or more impurities;
a selection valve arrangement, comprising a first chromatography device selection valve;
a first chromatography device comprising a first chromatography material comprising a support material functionalised with a ligand, wherein the ligand comprises an affinity group having a binding affinity for a biological target compound, an ion exchange group, or a multimodal group;
a second chromatography device, comprising a second chromatography material, which comprises a conditioning chromatography material;
a third chromatography device comprising a third chromatography material;
wherein the selection valve arrangement is configured to enable separation of biological target compounds from impurities by allowing a feed, comprising biological target molecules and one or more impurities, to continuously pass through the first, the second, and the third chromatography devices, wherein the first, the second, and the third chromatography devices are configured to be connected in series.

The present disclosure also provides use of the herein disclosed chromatography system for separation of a biological target compound from one or more impurities, wherein the biological target compound is selected from adeno-associated virus capsids, such as capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), adeno-associated virus serotype 10 (AAV10), adeno-associated virus serotype 11 (AAV11), adeno-associated virus serotype 12 (AAV12), or adeno-associated virus serotype 13 (AAV13), or a variant thereof.

Additionally, the present disclosure is directed to a method for separating adeno-associated virus capsids from one or more impurities, said adeno-associated virus capsids being fully packaged with genetic material, said method comprising:
a. Adding a feed comprising adeno-associated virus capsids fully packaged with genetic material, and one or more impurities, to a first chromatography device, comprising a first chromatography material comprising a support material functionalised with a ligand, wherein the ligand comprises an affinity group having a binding affinity for said adeno-associated virus capsids, an ion exchange group, or a multimodal group;
b. Eluting said adeno-associated virus capsids from the first chromatography device in at least one eluate fraction;
c. Adding the at least one eluate fraction comprising said adeno-associated virus capsids, obtained in step b, to a second chromatography device, comprising a second chromatography material comprising a conditioning chromatography material;
d. Obtaining said adeno-associated virus capsids in at least one flow-through fraction from the second chromatography device;
e. Adding the at least one flow-through fraction comprising said adeno-associated virus capsids, obtained in step d, to a third chromatography device, comprising a third chromatography material;
f. Obtaining said adeno-associated virus capsids from the third chromatography device in at least one eluate fraction;
wherein the feed is continuously passed through the first, the second and the third chromatography devices, to enable separation of said adeno-associated virus capsids from one or more impurities, wherein the first, the second and the third chromatography devices are connected in series.

Preferred aspects of the present disclosure are described below in the detailed description and in the dependent claims. It is noted that the present disclosure relates to all possible combinations of features recited in the claims.

### BRIEF DESCRIPTION OF THE DRAWINGS

These and other aspects of the present disclosure will now be described in more detail, with reference to the appended drawings showing embodiment(s) of the invention, in which:

FIG. 1 is a schematic outline of a chromatography system according to an embodiment.

FIG. 2 is a schematic outline of a chromatography system according to a further embodiment.

FIG. 3 is a flow chart outlining the steps of a method for separating adeno-associated virus capsids from impurities according to the present disclosure.

FIG. 4 shows chromatograms for separation of AAV5 full and empty capsids on different chromatography system set-ups, as described in Example 1 herein.

As illustrated in the figures, some features may be exaggerated for illustrative purposes and, thus, are provided to illustrate the general structures of embodiments of the present disclosure.

### DETAILED DESCRIPTION OF THE DISCLOSURE

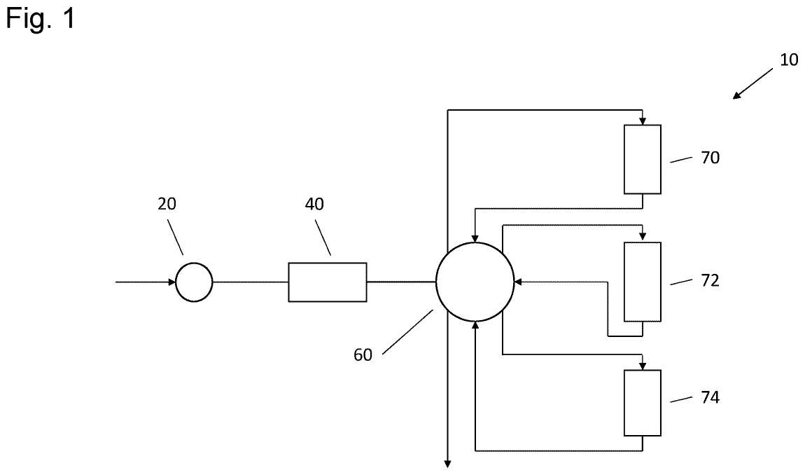

The present disclosure solves or at least mitigates the problems associated with existing chromatography systems and processes for separation of biological target compounds from impurities by providing, as illustrated in FIG. 1, a chromatography system 10 comprising:
a buffer valve arrangement 20 configured to allow independent control of a first buffer feed and a second buffer feed;
a pump arrangement 40 configured to supply a first buffer feed, a second buffer feed, and a feed comprising a biological target compound and one or more impurities;
a selection valve arrangement 60, comprising a first chromatography device selection valve (62);
a first chromatography device 70 comprising a first chromatography material comprising a support material functionalised with a ligand, wherein the ligand comprises an affinity group having a binding affinity for a biological target compound, an ion exchange group, or a multimodal group;
a second chromatography device 72, comprising a second chromatography material, which comprises a conditioning chromatography material;
a third chromatography device 74 comprising a third chromatography material;
wherein the selection valve arrangement is configured to enable separation of biological target compounds from impurities by allowing a feed, comprising biological target molecules and one or more impurities, to continuously pass through the first, the second, and the third chromatography devices, wherein the first, the second, and the third chromatography devices are configured to be connected in series.

The buffer valve arrangement 20 has at least one inlet (indicated by an arrow in FIG. 1) and a corresponding outlet for buffer feed, normally two or more inlets and corresponding outlets, for independent control of two or more buffer feeds.

The pump arrangement 40 includes at least one pump, optionally two or more pumps.

The selection valve arrangement 60 makes it possible to connect three chromatography devices in series and thereby to perform a three-step chromatography purification process in-line in the chromatography system. Interruptions and delays, traditionally encountered due to manual handling of various steps during the process, can hereby be avoided, which saves time and work efforts by users operating the equipment. The chromatography system may be completely set up and programmed before starting the purification process, after which the process may be started by one push of a button and allowed to continue to the end without further involvement by the user. The present disclosure may thus be said to provide a so-called “plug and play” chromatography system, meaning a system intended to work perfectly when first used or connected, without reconfiguration or adjustment by the user.

More particularly, the presently disclosed chromatography system 10 provides the following general advantages compared to a traditional chromatography system:
Connected desalting and pH adjustment step after affinity capture. This potentially saves time since no dilution is needed to reduce the conductivity and adjust the pH of the sample before polishing, which leads to lower volume of sample feed, and thereby a shorter loading time. Further, it gives full control over the sample conductivity to allow high performance full and empty AAV capsid separation;
No hold times;
No freeze-thaw cycles;
No sample conditioning (pH and conductivity) or sample handling between chromatography steps.

For purification of adeno-associated virus (AAV) vectors, an additional advantage is that the AAV vectors, which are sensitive to low pH for longer periods, are exposed to acidic conditions for shorter time (minutes vs hours). In addition, when purifying AAV vectors it has been shown that one step of tangential flow filtration may be avoided by making use of the herein disclosed chromatography system 10 compared to when using a conventional chromatography system for a traditional purification process.

The chromatography system 10 may be applied with various formats of chromatography devices and chromatography materials as described in more detailed elsewhere herein. Non-limiting examples of chromatography materials suitable for in-line connected purification of AAV vectors are combinations of membrane adsorbers, convection-based membranous structures comprising nanofibres, monoliths, and resins (beads). The herein disclosed chromatography system provides a significant time reduction (up to 70%) and simplified AAV purification process while achieving similar performance as a conventional purification set-up in terms of recovery and percentage of AAV capsids fully packaged with genetic material. It is surprising that a full capture and polishing process using such an in-line connected three-step purification process can be successfully implemented considering the combined chromatography challenge and the conductivity precision required for the polishing step.

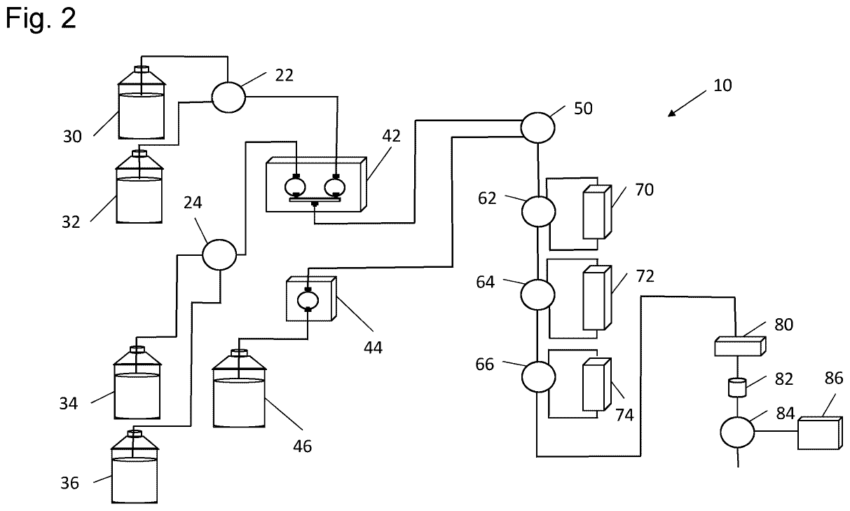

FIG. 2 illustrates a currently preferred, non-limiting embodiment of the chromatography system 10, in which the buffer valve arrangement consists of a first buffer selection valve 22, and a second buffer selection valve 24.

The first buffer selection valve 22 may be configured to control a first buffer feed, herein alternatively called buffer A, which is used when equilibrating a chromatography device before loading a sample feed comprising biological target compounds onto the chromatography device, and the second buffer selection valve 24 may be configured to control a second buffer feed, herein alternatively called buffer B, which is used when eluting the biological target compounds from the chromatography system 10.

More particularly, the first buffer selection valve 22 may be configured to control a feed of a first buffer 30 for equilibration of the first chromatography device 70, herein alternatively called buffer A1, and the first buffer selection valve 22 may further be configured to control a feed of a first buffer 32 for equilibration of the second chromatography device 72, herein alternatively called buffer A2.

Buffer 32 may also be used for equilibration of the third chromatography device 74. In cases where the biological target compounds are not binding to the second chromatography device but instead exiting the second chromatography device in flow-through, buffer 32 may also be used for passing the biological target compounds from the second chromatography device to the third chromatography device.

Further, the second buffer selection valve 24 may be configured to control a feed of a second buffer 34 for elution of biological target compounds from the first chromatography device 70, herein alternatively called buffer B1, and to control a feed of a second buffer 36 for elution of a biological target compound from the second chromatography device 72, herein alternatively called buffer B2.

Buffer B2 may optionally also be used for elution of a biological target compound from the third chromatography device 74. Alternatively, a separate buffer feed B3 may be used for elution of biological target compounds from the third chromatography device 74 (not shown).

The system 10 according to FIG. 2 further includes a pump arrangement consisting of a system pump 42 and a sample pump 44. The system pump 42 is configured to supply the feeds of buffer 30, buffer 32, buffer 34, and buffer 36. The sample pump 44 is configured to supply a feed of sample 46, comprising a biological target compound and one or more impurities.

The system 10 according to FIG. 2 also includes an injection valve 50. It is to be understood that any injection valve conventionally used in the art can be used.

According to the embodiment shown in FIG. 2, the selection valve arrangement consists of a first chromatography device selection valve 62, a second chromatography device selection valve 64 and a third chromatography device selection valve 66. The chromatography device selection valve 62 connects the first chromatography device 70, the second chromatography device selection valve 64 connects the second chromatography device 72, and the third chromatography device selection valve 66 connects the third chromatography device 74, wherein the three chromatography devices are connected in series in-line within the system 10.

However, it is contemplated that the selection valve arrangement 60 may consist of a single chromatography device selection valve 62, which connects the first, second and third chromatography devices 70, 72 and 74 in series (as shown in FIG. 1). Alternatively, the selection valve arrangement 60 may consist of a combination of a first chromatography device selection valve 62 and a second chromatography device selection valve 64 (not shown), which combination connects the first, second and third chromatography devices 70, 72 and 74 in series. It is to be understood that in embodiments where the number of chromatography device selection valves is lower than the number of chromatography devices, at least one of the selection valves can handle different flow paths, which makes it possible to perform a multi-step chromatography purification on a chromatography system including fewer selection valves than the number of chromatography devices.

A non-limiting example of a chromatography system which may be used is an AKTA pure chromatography system (Cytiva, Sweden). The AKTA pure system in its standard set-up has only one column valve (corresponding to a chromatography device selection valve of system 10). The AKTA pure system used in the experimental section herein has been modified by addition of two additional chromatography device selection valves, to obtain a system according to the embodiment shown in FIG. 2.

Another non-limiting example of a chromatography system which may be used is an NGC Chromatography System (BioRad, USA). It may be modified by addition of versatile valves, to obtain a system according to the embodiment shown in FIG. 2.

Non-limiting examples of chromatography device selection valves that may be part of the presently disclosed system 10 are so-called versatile valves, such as for example versatile valve V9-V (Cytiva, Sweden).

The chromatography system 10 according to FIG. 2 further includes a UV detector 80, a conductivity detector 82, an outlet valve 84 and a fraction collector 86, all of which are standard components of chromatography systems.

The herein disclosed chromatography system 10 is primarily intended for use in preparatory applications, for feed material of volumes ranging from a few mL to several hundreds of litres. The system may also be used for analytical applications. The distinguishing features of the system 10 provide more significant advantages when applied in large-scale processes than in small-scale processes.

The term “chromatography material” is used herein to denote a type of separation matrix.

The term “separation matrix” is used herein to denote a material comprising a support material to which one or more ligands comprising functional groups have been coupled. The functional groups of the ligand(s) bind compounds herein also called analytes, which are to be separated from a liquid sample and/or which are to be separated from other compounds present in the liquid sample. A separation matrix may further comprise a compound which couples the ligand(s) to the support material. The terms “linker”, “extender”, and “surface extender” may be used to describe such a compound, as further described below. Herein, the term “support material” may be used interchangeably with the term “support”.

The chromatography materials referred to herein may comprise a linker connecting the ligand to the support, i.e., the coupling of the ligand to the support is provided by introducing a linker between the support and ligand. The coupling may be carried out following any conventional covalent coupling methodology such as by use of epichlorohydrin; epibromohydrin; allyl-glycidylether; bis-epoxides such as butanedioldiglycidylether; halogen-substituted aliphatic substances such as di-chloro-propanol; and divinyl sulfone. Non-limiting examples of suitable linkers comprise vinyl sulfone, vinyl sulfone in combination with glycidol, polyethylene glycol (PEG) having 2-6 carbon atoms, carbohydrates having 3-6 carbon atoms, or polyalcohols having 3-6 carbon atoms.

Alternatively, the ligand may be coupled to the support via a longer linker molecule, also known as a “surface extender”, or simply “extender”. Extenders are well known in this field, and commonly used to sterically increase the distance between ligand and support. Extenders are sometimes denoted tentacles or flexible arms. For a more detailed description of possible chemical structures, see for example U.S. Pat. No. 6,428,707, which is hereby included herein by reference. In brief, the extender may be in the form of a polymer such as a homo- or a copolymer. Hydrophilic polymeric extenders may be of synthetic origin, i.e., with a synthetic skeleton, or of biological origin, i.e., a biopolymer with a naturally occurring skeleton. Typical synthetic polymers are polyvinyl alcohols, polyacrylamides and polymethacrylamides, polyvinyl ethers etc. Typical biopolymers are polysaccharides, such as starch, cellulose, dextran, agarose.

In this context, “ligand” is a molecule that has a known or unknown affinity for a given analyte and includes any functional group, or capturing agent, immobilized on its surface, whereas “analyte” includes any specific binding partner to the ligand. The term “ligand” may herein be used interchangeably with the terms “specific binding molecule”, “specific binding partner”, “capturing molecule” and “capturing agent”.

Herein, the molecules in a liquid sample which interact with a ligand are referred to as a “biological target compound” or an “analyte”. The term “biological target compound” encompasses various types of biological molecules and compounds. Non-limiting examples include plasmids, exosomes, mRNA, virus particles, and proteins, such as monoclonal antibodies.

Analytes of particular interest according to the present disclosure are adeno-associated virus capsids, more particularly adeno-associated virus capsids fully packaged with genetic material.

A “virus particle” is herein used to denote a complete infectious virus particle. It includes a core, comprising the genome of the virus (i.e., the viral genome), either in the form of ribonucleic acid (RNA) or deoxyribonucleic acid (DNA), and the core is surrounded by a morphologically defined shell. The shell is called a capsid. The capsid and the enclosed viral genome together constitute the so-called nucleocapsid. The nucleocapsid of some viruses is surrounded by a lipoprotein bilayer envelope. In the field of bioprocessing, for the purpose of producing viral vectors for various applications such as therapy, the genome of a virus particle is modified to include a genetic insert, comprising genetic material of interest. Modified virus particles are allowed to infect host cells in a cell culture and the virus particles are propagated in said host cells, after which the virus particles are purified from the cell culture by any means of separation and purification. Herein, a virus particle to be separated from a cell culture by the presently disclosed method may alternatively be referred to as a “biological target compound”, “target molecule”, or “target”. It is to be understood that “a virus particle” is intended to mean a type of virus particle and that the singular form of the term may encompass a large number of individual virus particles. Herein, the term “virus particle” may be used interchangeably with the terms “vector” and “capsid”, respectively, as further defined below.

The term “vector” is herein used to denote a virus particle, normally a recombinant virus particle, which is intended for use to achieve gene transfer to modify specific cell type or tissue. A virus particle can for example be engineered to provide a vector expressing therapeutic genes. Several virus types are currently being investigated for use to deliver genetic material (e.g., genes) to cells to provide either transient or permanent transgene expression. These include adenoviruses, retroviruses (γ-retroviruses and lentiviruses), poxviruses, adeno-associated viruses (AAV), baculoviruses, and herpes simplex viruses. Herein, the term “vector” may be used interchangeably with the terms “virus particle” and “capsid”, respectively.

The term “capsid” means the shell of a virus particle. The capsid surrounds the core of the virus particle, and normally should comprise a viral genome. A modified (recombinant) capsid, as produced in an upstream process of manufacturing, is supposed to comprise a complete viral genome, which genome includes genetic material of interest for one or more applications, for example of interest for various therapeutic applications. However, owing to low packaging efficiency, assembled capsids do not always contain any genetic material or only encapsidate truncated genetic fragments, resulting in so-called empty capsids and partially filled capsids, respectively. These capsids possess no therapeutic function, yet they compete for binding receptors during the cell-mediated processes. This may diminish the overall therapeutic efficacy and trigger undesirable immune responses. As a result, tracking these capsids throughout the production process is crucial to ensure consistent product quality and a proper dosing response (Xiaotong Fu et al, Analytical Strategies for Quantification of Adeno-Associated Virus Empty Capsids to Support Process Development, Human gene therapy methods, 2019, 30(4): 144-152). In up to 20-30% of a population of virus particles artificially produced in a cell culture, the capsid is only partially filled with genetic material. Further, in up to as much as 98% of artificially produced virus particles, the capsid does not comprise any part of the viral genome at all, i.e., it is empty. However, generally between 80% to 90% of artificially produced virus particles have empty capsids, and best cases currently achieve as little as 50% empty capsids.

Herein, the term “capsid” may be used interchangeably with the terms “vector” and “virus particle”, respectively. In the context of the present disclosure, a capsid may or may not comprise genetic material.

The term “genetic material of interest” is intended to mean genetic material which in the field of bioprocessing is considered relevant and valuable to get produced by viral replication and to purify such that it can be used in various applications, such as, but not limited to, therapeutic applications.

As a non-limiting example, genetic material of interest may comprise a therapeutically relevant genetic material, such as a therapeutically relevant nucleotide sequence.

The term “capsid fully packaged with genetic material” is herein used to denote a capsid which has been correctly produced (by the host cell), or in other words,
a capsid which comprises a complete viral genome, or in other words,
a capsid comprising 100% of its viral genome, or in other words,
a capsid comprising a functional viral genome.

The viral genome includes a genetic insert, comprising genetic material of interest, as defined elsewhere herein.

A capsid which comprises a complete viral genome may herein alternatively be called a “full capsid” or a “fully packaged capsid”. The terms “full capsid”, “fully packaged capsid”, and “capsid fully packaged with genetic material” may be used interchangeably throughout this text.

The term “capsid not fully packaged with genetic material” is herein used to denote a capsid which has not been correctly produced (by the host cell), or in other words, a capsid which does not comprise a complete viral genome, or in other words, a capsid which comprises less than 100% of its viral genome.

A capsid which is not fully packaged with genetic material is either partially filled with genetic material or is not filled with any genetic material at all.

The term “capsid not fully packaged with genetic material” encompasses the terms “partially filled capsid” and “empty capsid”, as defined below.

A “partially filled capsid” is herein defined as a capsid which comprises parts of its viral genome, such as defective parts of its viral genome, or in other words,
a capsid which comprises a partial viral genome, or in other words,
a capsid which comprises a non-complete viral genome, or in other words,
a capsid which comprises a defective viral genome, or in other words,
a capsid which comprises more than 0% and less than 100% of the complete viral genome, such as from about 1% to about 99%, such as from about 5% to about 95%, such as from about 10% to about 90%, or such as about 1%, about 5%, about 10%, about 20%, about 30%, about 40%, about 50%, about 60%, about 70%, about 80%, about 90%, about 95%, or about 99%, of the complete viral genome. Since a partially filled capsid is an incorrectly produced capsid, it is desirable to separate and remove as many as possible of the partially filled capsids from a population of capsids, before putting the population of capsids to use in its intended application, e.g., a therapeutic application.

Herein, a partially filled capsid may alternatively be called an “intermediate capsid”.

An “empty capsid” is herein defined as a capsid which does not comprise any part of its viral genome, i.e., which comprises 0% of its viral genome, or in other words, a capsid which is not filled with any genetic material at all. Thus, an empty capsid does not comprise any genetic material of interest. Consequently, it is desirable (and sometimes required, e.g., due to clinical regulations) to enrich the full capsids, i.e., to increase the percentage of full capsids at the expense of the percentage of partially filled capsids and empty capsids, by separating and remove as many as possible of the empty capsids from a population of capsids, before putting the population of capsids to use in its intended application, e.g., a therapeutic application.

The percentage of full capsids and empty capsids in a population of capsids can be estimated or analyzed with several methods known in the art. Some of these methods are briefly described below:
1: A260:280 in chromatogram will give an estimation of percentage full capsids present in peaks (ratio 1-1.5 indicate enriched in full capsids, ratio 0.5-0.7 is containing mainly empty capsids).
2. qPCR:ELISA ratio. qPCR quantifies viral genomes and ELISA quantifies total viral particles. A ratio of 2 assays with variation is less accurate and will be uncertain. Requires orthogonal analysis for confirmation (see below, 3, 4 or 5).
3. Analytical anion exchange separating full and empty capsids (A260:280 ratio and peak area to calculate the percentage). Accuracy dependent of peak definition.
4. Analytical ultracentrifugation (AUC). Detects and quantifies particles of different density (corresponding to full, partially filled, and empty capsids). This is currently known as the “golden standard” in the art. However, ultracentrifugation is not scalable and thus is not suitable for analysis of large-scale batches of capsids.
5. Transmission electron microscopy (TEM). Image analysis counting particles (full, partially filled, and empty capsids). May introduce artifacts from sample preparation.

Some methods for estimating or analyzing the percentage of full capsids and empty capsids in a population of capsids are described in more detail in Xiaotong Fu et al, Analytical Strategies for Quantification of Adeno-Associated Virus Empty Capsids to Support Process Development, Human gene therapy methods, 2019, 30(4): 144-152, which is hereby incorporated by reference herein.

Herein, the term “impurities” encompasses cell host debris, protein, genomic DNA, serum protein, some elements of medium, helper DNA, helper virus etc., which may be contained in the liquid sample in addition to the fully packaged adeno-associated virus capsids to be purified. The term “impurities” further includes not fully packaged adeno-associated virus capsids.

The term “surface” herein means all external surfaces and includes in the case of a porous support outer surfaces as well as pore surfaces.

The support material of the first chromatography material may comprise a membranous structure, nanofibres, a monolith, porous particles, non-porous particles, or expanded bed media.

A non-limiting example of a membranous structure is a Mustang® membrane (Pall Corporation, USA).

Another non-limiting example of a membranous structure is Fibro™ (Cytiva, Sweden), which is at the same time a non-limiting example of a support material comprising nanofibres. Fibro™ is a convection-based membranous structure comprising nanofibres made of cellulose or a cellulose derivative.

A non-limiting example of a monolith is CIMmultus® (Sartorius, Germany).

A non-limiting example of porous particles is Capto beads (Cytiva, Sweden), which are substantially spherical particles having a diameter of approx. 90 μm.

A non-limiting example of expanded bed media is STREAMLINE resins (Cytiva, Sweden).

As mentioned further above, the ligand of the first chromatography material may comprise an affinity group having a binding affinity for a biological target compound, an ion exchange group (more particularly, an anion exchange group or a cation exchange group), or a multimodal group.

Where the ligand of the first chromatography material comprises an affinity group, it may have a binding affinity for adeno-associated virus capsids. Herein, such a ligand is alternatively called an AAV vector ligand. The AAV vector ligand may be a peptide or polypeptide, including an antibody or an antibody fragment, an oligonucleotide, such as DNA or RNA, such as an aptamer. The AAV vector ligand may be a camelid antibody or antibody fragment. AAV vector binding ligands are known in the art. For example, POROS CaptureSelect AAVX chromatography material (ThermoFisher Scientific, USA) has demonstrated binding reactivity towards a set of AAV serotypes that includes AAV1 to AAV8, and AAVrh10. Other examples of chromatography materials incorporating affinity ligands include AVIPure® AAV2 affinity resin, AVIPure® AAV8 affinity resin and AVIPure® AAV9 affinity resin (Avitide/Repligen, USA). As another example, Capto AVB and AVB Sepharose High Performance (Cytiva, Sweden) are affinity chromatography materials with proven affinity for adeno associated viruses from subclasses 1, 2, 3, and 5.

Where the ligand of the first chromatography material comprises an anion exchange group, it may be a strong or partially strong anion exchange group, more particularly a quaternized amine group. A quaternary amine group is a strong anion exchange group, which is always positively charged irrespective of to which pH it is subjected. For DEAE-based types of chromatography materials, the degree of quaternization of the amine group may vary among the amine groups included in a chromatography material. A degree of quaternization of the amine group of from about 12% to about 100% globally in a chromatography material is generally considered to result in a chromatography material which behaves like a strong, or at least partially strong, anion exchange chromatography material since these at least 12% of all amine groups are always charged.

More particularly, where the ligand of the first chromatography material comprises a strong or partially strong anion exchange group, the ligand may be defined by Formula I:

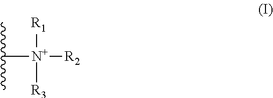

wherein R₁ is selected from H and C1-C3 alkyl, and R₂ and R₃ are independently selected from H, C1-C3 alkyl, CH2OH, and CH2CHOHCH3, preferably wherein each of R1, R2, and R3 is CH3.

The wavy moiety represents the support material, including the linker. The ligand may be attached to a carbon atom of the linker.

There are currently available chromatography materials comprising a ligand defined by Formula I, wherein each of R1, R2, and R3 is CH3; e.g., a chromatography material made available under the name Capto Q (Cytiva, Sweden). Capto Q further comprises dextran as surface extender. Capto Q is a non-limiting example of a strong anion exchange chromatography material having about 100% quaternized amine groups.

Where the ligand of the first chromatography material comprises a cation exchange group, it may for example be a sulfonate group. A non-limiting example is Capto S (Cytiva, Sweden). Another non-limiting example is CIMmultus® SO3 (Sartorius, Germany)).

Where the ligand of the first chromatography material comprises a multimodal group, the multimodal group may for example be a multimodal weak cation exchange group or a multimodal weak anion exchange group. A non-limiting example is Capto MMC (Cytiva, Sweden), which is a multimodal weak cation exchanger.

The second chromatography material is a conditioning chromatography material, wherein the term “conditioning chromatography material” is intended to mean that it conditions or prepares a sample feed or solution, which is loaded onto and being passed through the chromatography material, for subsequent purification steps and/or final formulation of a product. The conditioning may for example include or consist of desalting, i.e., decreasing of the salt concentration, of the sample feed or solution. Alternatively, conditioning may include or consist of increasing the salt concentration of the sample feed or solution. Alternatively, or additionally, conditioning may include or consist of changing the pH of the sample feed or solution, i.e., decreasing or increasing the pH.

When the conditioning consists of desalting, the conditioning chromatography material may suitably comprise a size exclusion chromatography material. Salt is retarded by the size exclusion material while the biological target compounds, which are much bigger than salt molecules, pass through the size exclusion material without retardation and thus are obtained in flow-through fractions from the second chromatography material.

The support material of the third chromatography material may comprise a membranous structure, nanofibres, a monolith, porous particles, non-porous particles, or expanded bed media. Such support materials have been described in more detail elsewhere herein.

The ligand of the third chromatography material may comprise an anion exchange group or a multimodal group.

Where the ligand of the third chromatography material comprises an anion exchange group, it may comprise a strong or partially strong anion exchange group, as defined and described in more detail above in relation to the first chromatography material.

More particularly, where the ligand of the third chromatography material comprises a strong or partially strong anion exchange group, the ligand may be defined by Formula I:

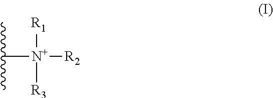

wherein R₁ is selected from H and C1-C3 alkyl, and R₂ and R₃ are independently selected from H, C1-C3 alkyl, CH2OH, and CH2CHOHCH3, preferably wherein each of R₁, R₂, and R₃ is CH3.

The wavy moiety represents the support material, including the linker. The ligand may be attached to a carbon atom of the linker.

A non-limiting example of a chromatography material comprising a ligand defined by Formula I, wherein each of R1, R2, and R3 is CH3, is Capto Q (Cytiva, Sweden).

Another non-limiting example of a chromatography material comprising a quaternary amine ligand is the monolithic CIMmultus® QA (Sartorius, Germany).

Where the ligand of the third chromatography material comprises a strong or partially strong anion exchange group, it may alternatively be defined by Formula II:

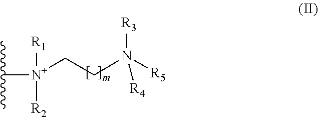

wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3; provided that if m is 1, the ligand of the strong, or partially strong, anion exchange chromatography material is defined by the following Formula II:

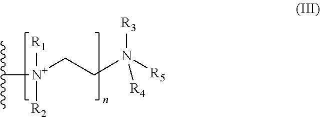

wherein n is an integer of from 0 to 3;
provided that if n is 0, R₃ and R₄ are independently selected from C1-C3 alkyl, and R₅ is hydrogen or CH2CHOHCH3.

As a non-limiting example, the ligand is defined by Formula III and comprises a combination of two or more of the following structures (i)-(iv):
(i) n is 0; R₃ and R₄ are ethyl; and R₅ is hydrogen or CH2CHOHCH3;
(ii) n is 1; R₁, R₂, R₃, R₄ are ethyl; and R₅ is hydrogen or CH2CHOHCH3;
(iii) n is 2; each R₁ and R₂ is ethyl; R₃ and R₄ is ethyl; and R₅ is hydrogen or CH2CHOHCH3;
(iv) n is 3; each R₁ and R₂ is ethyl; R₃ and R₄ is ethyl; and R₅ is hydrogen or CH2CHOHCH3.

One currently available chromatography material comprising a ligand defined by Formula III and comprising a combination of the above-mentioned structures (i)-(iv) is the chromatography resin called Capto DEAE (Cytiva, Sweden). Capto DEAE further comprises dextran as surface extender. Capto DEAE is a non-limiting example of a strong, or partially strong, anion exchange chromatography material having a degree of quaternization of the amine groups of about 15%.

Where the ligand of the third chromatography material comprises an anion exchange group, it may alternatively comprise a weak anion exchange group. In contrast to the above-defined quaternized amine groups, almost all other ionic exchange groups are weak, i.e., their charge varies from fully charged to not charged within a reasonable range of pH used (such as pH 2-11) and having a neutral charge (same amount of + and − charges) at pl.

More particularly, where the ligand of the third chromatography material comprises a weak anion exchange group, the ligand may be defined by Formula IV:

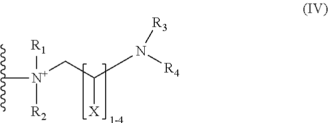

wherein X, for each occurrence independently, is selected from H, OH and a C₁₋₃ group, and R₁, R₂, R₃ and R₄ are independently selected from H and a C₁₋₃ group, wherein a C₃ group is straight or branched, wherein a C₁₋₃ group comprises groups independently selected from OH, O—C₁₋₂, S—C₁₋₂, NH, NHR, and NR₂, wherein R is selected from H and a C₁₋₃ group.

Non-limiting examples of ligands defined by Formula IV are N,N,N′-triethylethylenediamine, diethylenetriamine, N,N′-dimethylethylenediamine, N-methylethylenediamine, 1,3-diaminopropane, 1,3-diamino-2-hydroxypropane, 2-methyl-1,3-propanediamine and N,N-diethylethylenediamine. A currently preferred ligand comprising a weak anion exchange group is N,N-diethylethylenediamine.

Where the ligand of the third chromatography material comprises a multimodal group, the multimodal group may for example be a multimodal weak cation exchange group or a multimodal weak anion exchange group. A non-limiting example is Capto MMC (Cytiva, Sweden), which is a multimodal weak cation exchanger.

The present disclosure further provides use of the above-described chromatography system 10, including the above-described first, second and third chromatography materials, for separation of a biological target compound from one or more impurities, wherein the biological target compound is selected from adeno-associated virus capsids, such as capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), adeno-associated virus serotype 10 (AAV10), adeno-associated virus serotype 11 (AAV11), adeno-associated virus serotype 12 (AAV12), or adeno-associated virus serotype 13 (AAV13), or a variant thereof.

The term “variant” in relation to an adeno-associated virus (AAV) serotype as listed above, is intended to mean a modified or engineered AAV, in which the capsid structure has been modified to improve clinical performance, for example towards a specific target organ. As a non-limiting example, an AAV8 variant comprises capsid parts of AAV8 and may additionally comprise capsid parts of other AAV serotypes than AAV8, such as AAV5. However, an AAV8 variant as referred to herein must retain a significant structural similarity to a non-modified AAV8 capsid, such as retaining at least 50%, such as 60%, 70%, 80%, or 90%, of the external surface structure of a non-modified AAV8 capsid. This applies equally to a variant of AAV serotype 1, 2, 3, 4, 5, 6, 7, 9, 10, 11, 12, and 13 as compared to a non-modified AAV serotype 1, 2, 3, 4, 5, 6, 7, 9, 10, 11, 12, and 13, respectively. Further, as a non-limiting example, in the context of purification or separation of a variant of AAV8, a “variant” is herein defined as an adeno-associated virus which has a functionally equivalent binding capacity to the ligand of a specified chromatography material, compared to the binding capacity of the original AAV8 to said specified chromatography material. This applies equally to a variant of AAV serotype 1, 2, 3, 4, 5, 6, 7, 9, 10, 11, 12, and 13, as compared to the original AAV serotype 1, 2, 3, 4, 5, 6, 7, 9, 10, 11, 12, and 13, respectively. The specified chromatography material may, for example, be a chromatography material as disclosed in more detail elsewhere herein. A variant of an adeno-associated virus may for example be obtained by spontaneous mutation, or by engineered modification (i.e., obtained by human interaction), of one or more nucleotides of the genome of the adeno-associated virus.

In particular, the adeno-associated virus capsids may be selected from a group consisting of serotypes AAV2, AAV5, AAV8, and AAV9, or a variant of any one of said serotypes.

Currently preferred, non-limiting examples of the first chromatography material include:
(i) a support material in the form of porous particles functionalised with a ligand comprising an affinity group which has a binding affinity for adeno-associated virus capsids;
(ii) a support material in the form of a convection-based membranous structure comprising a non-woven web of polymer nanofibres functionalised with a ligand comprising an affinity group which has a binding affinity for adeno-associated virus capsids;
(iii) a support material in the form of porous particles functionalised with a ligand comprising a multimodal weak cation exchanger; and
(iv) a support material in the form of porous particles functionalised with a ligand comprising a sulfonate group.

Currently preferred, non-limiting examples of the third chromatography material include:
i. a support material in the form of porous particles functionalised with a ligand defined by Formula I, wherein each of R1, R2, and R3 is CH3; optionally wherein the ligand is connected to the support material by dextran; optionally wherein the adeno-associated virus capsids to be separated are capsids of AAV2, AAV5, AAV8, or AAV9;
ii. a support material in the form of a convection-based membranous structure comprising a non-woven web of polymer nanofibres functionalised with a ligand defined by Formula I, wherein each of R1, R2, and R3 is CH3; optionally wherein the adeno-associated virus capsids to be separated are capsids of AAV2, AAV5, or AAV8;
iii. a support material in the form of porous particles or a convection-based membranous structure comprising a non-woven web of polymer nanofibres, functionalised with a diethylethanolamine ligand; optionally wherein the adeno-associated virus capsids to be separated are capsids of AAV2, AAV5, or AAV8; and
iv. a support material in the form of porous particles or a convection-based membranous structure comprising a non-woven web of polymer nanofibres, functionalised with a N,N-diethylethylenediamine ligand; optionally wherein the adeno-associated virus capsids to be separated are capsids of AAV2, AAV5, or AAV8.

Further, when using the chromatography system 10 for separation of AAV capsids, it is currently preferred to select the first chromatography material from the options (i)-(iv) listed above and to select the third chromatography material from the options i-iv listed above. The system 10 may thus suitably comprise any combination among said options of the first chromatography material and said options of the third chromatography material. In addition, the second chromatography material, which is a conditioning chromatography material, is currently preferred a desalting chromatography material.

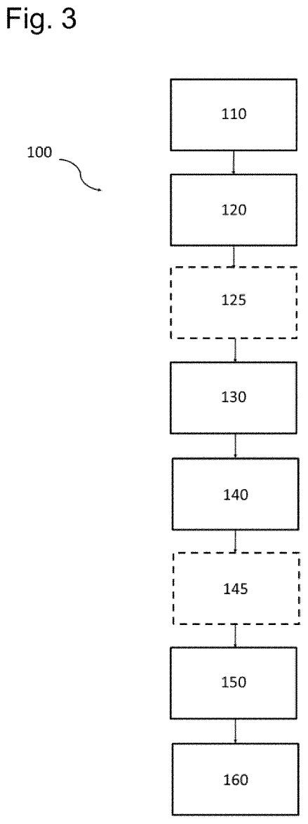

The present disclosure additionally provides, as illustrated in FIG. 3, a method 100 for separating adeno-associated virus capsids from one or more impurities, said adeno-associated virus capsids being fully packaged with genetic material, said method comprising:
a. Adding 110 a feed comprising adeno-associated virus capsids fully packaged with genetic material, and one or more impurities, to a first chromatography device 70, comprising a first chromatography material comprising a support material functionalised with a ligand, wherein the ligand comprises an affinity group having a binding affinity for said adeno-associated virus capsids, an ion exchange group, or a multimodal group;
b. Eluting 120 said adeno-associated virus capsids from the first chromatography device in at least one eluate fraction;
c. Adding 130 the at least one eluate fraction comprising said adeno-associated virus capsids, obtained in step b, to a second chromatography device 72, comprising a second chromatography material comprising a conditioning chromatography material;
d. Obtaining 140 said adeno-associated virus capsids in at least one flow-through fraction from the second chromatography device;
e. Adding 150 the at least one flow-through fraction comprising said adeno-associated virus capsids, obtained in step d, to a third chromatography device 74, comprising a third chromatography material;
f. Obtaining 160 said adeno-associated virus capsids from the third chromatography device in at least one eluate fraction;
wherein the feed is continuously passed through the first, the second and the third chromatography devices, to enable separation of said adeno-associated virus capsids from one or more impurities,
wherein the first, the second, and the third chromatography devices are connected in series.

The term “eluate” is used in its conventional meaning in this field, i.e., the part(s) of a liquid sample which are eluted from a chromatography column after having loaded the liquid sample onto the chromatography column.

It is to be understood that the first, the second, and the third chromatography materials, and the corresponding chromatography devices 70, 72 and 74, referred to in the method 100 are as defined and exemplified in detail further above in connection with the description of the chromatography system 10.

In the method 100, the chromatography material referred to in steps (a) and (b) may be referred to as a capture chromatography material, meaning that the chromatography material is applied in a capture step, which in the context of liquid chromatography refers to the initial step(s) of a separation procedure. Herein, the capture step performed in steps (a) and (b) of the method 100 achieves a significant purification of the biological target compound from soluble impurities.

Additional steps such as clarification and filtration (e.g., tangential flow filtration) may be performed prior to steps (a) and (b).

Further, the object of steps (c) and (d) of the method 100 is to condition or prepare the biological target compound for the conditions required to perform the subsequent purification steps (e) and (f). More particularly, for separation of AAV vectors, it is crucial to decrease the conductivity of the sample to achieve a good separation of full and empty AAV capsids in the subsequent purification steps. This in-line connected desalting performed in steps (c) and (d) is an important part of the present disclosure, helping to achieve good separation results while reducing the process time.

Conventionally, a desalting step would be performed in a separate device, not connected in-line to the other chromatography devices.

In the method 100, the chromatography material referred to in steps (e) and (f) may be referred to as a polishing chromatography material, meaning that the chromatography material is applied in a polishing step.

The term “polishing step” refers in the context of liquid chromatography to a final purification step, wherein trace impurities are removed to leave an active, safe product. Impurities removed during the polishing step are often conformers of the target molecule, i.e., forms of the target molecule having particular molecular conformations, or suspected leakage products. A polishing step may alternatively be called “secondary purification step”.

In the method 100 illustrated in FIG. 3, the flow rate used is dependent on the type of chromatography material used and the dimensions of the chromatography material or the chromatography device containing said chromatography material), and the residence time chosen.

For example, a much higher flow rate can be applied to convection-based membranous support material, such as Fibro™, than to porous particles, such as Capto beads.

Further, as appreciated by persons skilled in the art, different buffers used in the method 100 are selected based on which chromatography materials are applied and which biological target compounds are to be purified.

In steps (a) and (b) of the method, the pH of the buffer used may vary depending on which type of ligand is used. For affinity ligands, the buffer may for example have a pH around 7-8 for binding (step (a)) and pH 1.5-4 for elution (step (b)). For cation exchange ligands and for multimodal cation exchange ligands, the buffer pH may be around 4.5-6.5 for both binding (step (a)) and elution (step (b)).

Steps (e) and (f) of method 100 may comprise applying a buffer having a pH of from about 6.0 to about 10.5, such as from about 7.0 to about 10.0, such as from about 7.5 to about 9.5. According to non-limiting examples, a pH of about 9.5 may be applied for a chromatography material comprising a ligand defined by Formula I. Further, a pH of about 7.5 may be applied for a chromatography material comprising a ligand defined by Formula II or Formula III. Said buffer is suitably selected from buffers generally recommended for anion exchange chromatography and may for example comprise tris(hydroxymethyl)amino-methane (i.e., Tris), 1,3-bis(tris(hydroxymethyl)methylamino) propane (i.e., bis-Tris propane), triethanolamine, N-methyldiethanolamine, diethanolamine, 1,3-diaminopropane, or ethanolamine.

The buffer applied in step (f) additionally comprises a compound which may help eluting capsids bound to the chromatography material. This compound is not present in the buffer applied in step (e). Non-limiting examples of such a compound is a salt, such as a salt of a monovalent metal ion.

Non-limiting examples are NaCl, LiCl, KCl, or other equivalent metal salt suitable to use for salt elution, as is well known in the art. Further, step (f) may comprise applying a gradient of such a compound to improve elution of the adeno-associated virus capsids fully packaged with genetic material from the chromatography material. Such a gradient may be a linear gradient or a step gradient, or a combination thereof. In a currently preferred embodiment, step (f) includes applying a step gradient of increased salt concentration, eluting not fully packaged capsids at a first, lower salt concentration and eluting fully packaged capsids at a second, higher salt concentration.

The buffer applied in step (f) may additionally comprise a compound which improves separation between capsids fully packaged with genetic material and capsids not fully packaged with genetic material. This compound may or may not be present in a buffer applied in step (e). Said compound which improves separation may for example be selected from a carbohydrate, a divalent metal ion, and a detergent.

As described above, the object of steps (c) and (d) is to condition or prepare the biological target compounds for the condition required to perform the subsequent steps (e) and (f). Thus, the buffer used in steps (c) and (d) will normally be the same as the buffer used in step (e). In general, the buffer 34 used in step (b) of the method, for elution of the capsids from the first chromatography material, is not suitable for binding of the capsids to the third chromatography material. Hence, buffer 32, used in step (c) and step (e), for equilibration and loading of a feed onto the second chromatography material and onto the third chromatography material, will be a different buffer than buffer 34.

A person skilled in the art is able to choose a suitable concentration for any one of the above-listed buffers.

A currently preferred, non-limiting example of a suitable combination of buffers used for separation of AAV capsids is:
Buffer 30 (buffer A1): 20 mM Tris-HCl pH 7.5, 0.5 M NaCl
Buffer 32 (buffer A2): 20 mM bis-Tris-propane (BTP) pH 9.0, 2 mM MgCl₂
Buffer 34 (buffer B1): 100 mM Glycine pH 2.5
Buffer 36 (buffer B2): 20 mM bis-Tris-propane (BTP).

The method 100 suitably comprises applying a selection valve arrangement 60 configured to enable the separation of AAV capsids from impurities, by allowing the feed to continuously pass through the first, the second, and the third chromatography devices 70, 72 and 74. The selection valve arrangement comprises a first chromatography device selection valve 62, and may optionally further comprise a second chromatography device selection valve 64, and optionally also a third chromatography device selection valve 66, as described in more detail elsewhere herein.

As seen in FIG. 3, the method 100 may further comprise an optional step of equilibrating 125 the second chromatography material before step c to conditions required for the adeno-associated virus capsids to be obtained in step d, and to be obtained in step f.

Similarly, the method 100 may further comprise an optional step of equilibrating 145 the third chromatography material before step e to conditions required for the adeno-associated virus capsids to be obtained in step f.

The herein disclosed method 100 may be a preparative method (preferably) or an analytical method.

The distinguishing features of the presently disclosed chromatography system 10 makes it possible to equilibrate the second chromatography material and/or the third chromatography material in-line while the method 100 is running, as opposed to equilibrating them separately, as isolated steps, thereby interrupting the continuous flow of feed in-line, within the system 10. This is advantageous since it enables quick neutralisation of pH and reduction of conductivity of the AAV vector sample and contributes to reducing the overall time required to perform the process.

Additional reduction of time can be achieved since loading of the different chromatography devices within the system 10 may be done partly simultaneously. For example, it is not required to wait until the sample feed has passed through the second or third device before more sample is loaded into the first device, etc.

The capsids to be separated from impurities by using the method 100 may be capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), adeno-associated virus serotype 10 (AAV10), adeno-associated virus serotype 11 (AAV11), adeno-associated virus serotype 12 (AAV12), or adeno-associated virus serotype 13 (AAV13), or a variant thereof.

In particular, the adeno-associated virus capsids may be selected from a group consisting of serotypes AAV2, AAV5, AAV8, and AAV9, or a variant of any one of said serotypes.

The term “variant” has been defined elsewhere herein.

It is to be understood that the present disclosure is not restricted to the below-described exemplifying embodiments thereof and that several conceivable modifications of the present disclosure are possible within the scope of the following claims. In the claims, any reference signs placed between parentheses shall not be construed as limiting the claim. Use of the verb “to comprise” and its conjugations does not exclude the presence of elements or steps other than those stated. The article “a” or “an” preceding an element does not exclude the presence of a plurality of such elements.

### EXPERIMENTAL SECTION

### Example 1: AAV5 Full and Empty Capsid Separation by in-Line Connected Affinity Capture, Desalting, and Polishing Chromatography

A three-step chromatographic process was performed in one run by use of an AKTA pure™ chromatography system (Cytiva, Sweden), which has been modified by addition of two versatile valves. Two alternative in-line connected set-ups for capture and polishing of AAV5 using either bead resin columns or nanofibre membrane adsorber units were evaluated. A hands-free walk away set-up with one desalting column in between the affinity capture device and the polishing column was used to condition the sample and ensure low conductivity load for high performance full and empty capsid separation.

### 1^{st} Set-Up

The modified ÄKTA pure™ chromatography system comprised:
a 1^{st} chromatography device selection valve, herein called column valve 1 (CV1),
a 1^{st} column comprising affinity capture bead material, 1 mL Capto AVB HiTrap (Cytiva, Sweden),
a 2^{nd} chromatography device selection valve, herein called versatile valve 1 (VV1) which has been added compared to a standard AKTA instrument,
a 2^{nd} column comprising a desalting material, 50 mL HiPrep 26/10 Desalting (Cytiva, Sweden),
a 3^{rd} chromatography device selection valve, herein called versatile valve 2 (VV2), which has been added compared to a standard AKTA instrument, and
a 3^{rd} column comprising polishing bead material comprising anion exchange ligands, 1 mL Capto Q HiTrap (Cytiva, Sweden).

The three in-line connected columns were equilibrated separately before sample loading. Clarified, concentrated (by way of tangential flow filtration), and buffer-exchanged material AAV5-GFP (tangential flow filtration (TFF), 10× ultrafiltration and 5× diafiltration) with a titer of approx. 1×10¹² viral particles (VP)/mL in total, of which 2.6×10¹¹ viral particles including viral genomes (VG)/mL (I.e., approx. 26% full capsids), was loaded onto the first column and the protocol below in Table 1 was used.

TABLE 1
Protocol (Unicorn ™ software) for 1^{st} set-up.

| | Volume | Flowrate | % | | | | |
|---|---|---|---|---|---|---|---|
| Phase | (ml) | (ml/min) | B | Inlets | CV1 | VV1 | VV2 |
| Equilibration | 0.5 | 1.25 | 0 | A1/B1 | on | off | off |
| Sample | 50 | 0.25 | 0 | A1/B1 | on | off | off |
| application | | | | | | | |
| Column wash | 15 | 2 | 0 | A1/B1 | on | off | off |
| Capto AVB | | | | | | | |
| Elution from | 5 | 1.25 | 100 | A1/B1 | on | on | off |
| CV1 to VV1 | | | | | | | |
| Elution from | 30 | 5 | 0 | A2/B2 | off | on | on |
| VV1 to VV2* | | | | | | | |
| Elution VV2- | 25 | 1 | 33 | A2/B2 | off | off | on |
| empty capsids | | | | | | | |
| Elution VV2- | 5 | 1 | 100 | A2/B2 | off | off | on |
| full capsids | | | | | | | |

*Pump wash A2/B2 was initiated

### 2^{nd} Set-Up

The modified AKTA chromatography system comprised:
a 1^{st} chromatography device selection valve, herein called column valve 1 (CV1),
a 1^{st} chromatography device, more particularly a membrane adsorber unit comprising affinity capture nanofibre material, 0.4 mL Fibro AVB prototype,
a 2^{nd} chromatography device selection valve, herein called versatile valve 1 (VV1) which has been added compared to a standard AKTA instrument,
a 2^{nd} chromatography device, more particularly a column comprising a desalting material, 50 mL HiPrep 26/10 Desalting (Cytiva, Sweden),
a 3^{rd} chromatography device selection valve, herein called versatile valve 2 (VV2), which has been added compared to a standard AKTA instrument, and
a 3^{rd} chromatography device, more particularly a membrane adsorber unit comprising polishing nanofibre material, 0.4 mL Fibro anion exchange prototype (Fibro-NG-DAX IC 162 μmol/mL).

The three in-line connected chromatography devices were equilibrated separately before sample loading. Clarified AAV5-GFP material with a titer of approx. 1.3×10¹¹ VP/mL and 3.0×10¹⁰ VG/mL (approx. 23% full capsids) was loaded onto the first chromatography device and the protocol below in Table 2 was used.

TABLE 2
Protocol (Unicorn ™ software) for 2^{nd} set-up.

| | Volume | Flowrate | % | | | | |
|---|---|---|---|---|---|---|---|
| Phase | (ml) | (ml/min) | B | Inlets | CV1 | VV1 | VV2 |
| Equilibration | 0.5 | 10 | 0 | A1/B1 | on | off | off |
| Sample | 3500 | 10 | 0 | A1/B1 | on | off | off |
| application | | | | | | | |
| Column wash | 15 | 10 | 0 | A1/B1 | on | off | off |
| Capto AVB | | | | | | | |
| Elution from | 5 | 5 | 100 | A1/B1 | on | on | off |
| CV1 to VV1 | | | | | | | |
| Elution from | 30 | 5 | 0 | A2/B2 | off | on | on |
| VV1 to VV2* | | | | | | | |
| Elution VV2- | 25 | 10 | 2 | A2/B2 | off | off | on |
| empty capsids | | | | | | | |
| Elution VV2- | 5 | 10 | 100 | A2/B2 | off | off | on |
| full capsids | | | | | | | |

*Pump wash A2/B2 was initiated

Buffer system used for both 1^{st} and 2^{nd} set-up:
A1: 20 mM Tris-HCl pH 7.5, 0.5 M NaCl
A2: 20 mM bis-Tris-propane (BTP) pH 9.0, 2 mM MgCl₂
B1: 100 mM Glycine pH 2.5
B2: 20 mM BTP

For the 2^{nd} set-up, the affinity capture Fibro prototype and the polishing Fibro prototype were prepared by producing a support material comprising a non-woven web of cellulose acetate nanofibres and functionalising it with ligands, as follows.

### Preparation of Affinity Capture Fibro Prototype:

### Sheet Production:

The support material may be produced as described in WO2018/011599, or as to produce a laminated non-woven sheet of fibres. A solution of cellulose acetate (CA) with a relative molecular mass of 29,000 g/mol was dissolved in a binary mixture of glacial acetic acid and ethanol in a 3:1 ratio (so-called primary CA solution). Polyethethylene oxide dissolved in deionised (DI) water to a concentration of 5% was then added to the primary CA solution in a quantity of 1.2% of the total volume of CA prior to electro spinning to produce fibres with diameters ranging between 300-600 nm. Optimised conditions for nanofibre production can be found in, for example, 0. Hardick, et al, J. Mater. Sci. 46 (2011) 3890. Sheets of approximately 20 g/m2 material were layered and subjected to a combined heating and pressure treatment.

The support material thus formed has a mean flow pore size of 0.1-2.0 μm, which may be measured using bubble point porometry, e.g., by use of a Porolux 100 porometer (IB-FT GmbH, Germany).

Through varying a combination of standard electrospinning parameters and reducing the number of fibre layers, mean flow pore sizes across this range can be reliably obtained.

### CA Pre-Washing:

35×sheets of the CA material (100×155 mm²) were sandwiched between gauze and loaded into a flow reactor. The material was washed by recirculation of 5 L DI water for 20 min. The reactor was emptied, and the washing process was repeated a further 2 times, with material being stored overnight in the final wash if necessary.

### Glycidol Step:

In an 8 L beaker, KOH (265 g) was added to DI water (4723 mL). The solution was stirred vigorously. Upon complete dissolution, glycidol (1350 mL or 675 mL, dependent on 100% or 50% glycidol base matrix, respectively) was added and stirred vigorously for 4 min. The reactor was emptied from the washing water and the KOH/glycidol solution was added to the reactor. The recirculating pump was started. A reaction temperature profile of 20° C. was used and the solutions continuously flown through the membranes to prevent the reaction temperature exceeding 20° C. After 6-7 h of recirculation, the reacting solution was removed, and the material was washed by recirculation of 5 L of DI water for 20 min and then emptied. The washing process was repeated at least 3 times (or as many times as necessary until the final pH is neutral). The sheets were stored in DI water overnight.

### Saponification Step:

Material made and referred to as 0% glycidol is known as regenerated cellulose (RC) and is synthesised in a saponification reaction where the terminal acetate groups on the cellulose acetate backbone are cleaved, to leave alcohol groups. This step occurs in substitution of the glycidol step and is followed by a divinyl sulfone (DVS) step (see below). In an 8 L beaker, KOH (132 g) was added to DI water (3149 mL), with EtOH (1574 mL). The solution was stirred vigorously until complete dissolution. The washing water was emptied from the flow reactor and KOH/EtOH solution added. The recirculating pump was started and run for 6 h at 22-24° C. Thereafter, the reaction mixture was removed, and 5 L DI water was added to the reactor. The recirculating pump was started and run for 20 min. The washing water was removed, and the water washing process was repeated a further 3 times.

Divinyl sulfone (DVS) Step:

The flow reactor was emptied from the washing water. In an 8 L beaker, Na2CO3 (316 g) was added to DI water (4211 mL). The solution was vigorously stirred until complete dissolution. Acetonitrile (1258 mL) was added under vigorous stirring. The solution was added to the flow reactor. The recirculating pump was started for 4 min, before DVS (1350 mL) was added carefully in one portion to the reaction vessel. After 6 h of recirculation, the reaction mixture was removed. A 1:1 acetone/DI water (5 L) was added to the flow reactor and the recirculating pump was started and ran for 20 min. The washing solution was removed. The acetone/water washing process was repeated a further 3 times. DI water (5 L) at 22-24° C. was added to the flow reactor and the recirculating pump was started and ran for 20 min. The washing water was removed, and the water washing process was repeated once.

### Immobilisation of Affinity Ligand:

An AAV vector ligand of choice can be immobilized to the support material. In this example, a spin filtered solution of AAV binding ligand having a concentration of 2.5 mg/ml was used for coupling. A coupling solution of 3.0 M (NH4)2SO4, 0.1 M NaHCO3 was prepared and adjusted to pH 9. A sheet of DVS treated support material was placed into a sealable container (155×105 mm²) and an amount of ligand solution at the desired concentration (0.5-6.0 mg/mL) added with an amount of coupling solution, either 17 or 34 mL, to make up the desired total volume, either 25 mL or 50 mL. The container was sealed and placed on an orbital shaker for 16 h at 22-24° C. After this time, the supernatant was collected. All sheets were washed with DI water for 20 min. This was repeated a further 3×, collecting the wash supernatant each time for later quantification of the immobilisation efficiency.

### Blocking:

Either blocking with ethanolamine or blocking with thioglycerol was carried out.

Blocking with ethanolamine: A blocking solution of 0.3 M ethanolamine was adjusted to pH 9 and 25 mL dispensed onto each sheet. The containers were sealed and placed on an orbital shaker for 16 h at 22-24° C. After this time, the blocking solution was discarded and sheets were washed with DI water for 20 min. This was repeated once. Sheets were washed with PBS adjusted to pH 2.0 for 20 min, followed by PBS at pH 7.4. This two-step process was repeated once, followed by 2× DI water washes for 20 min each.

Blocking with thioglycerol: A blocking solution of 0.288M thioglycerol, 0.1 M Na2HPO4·12H2O, 0.001 M EDTA was adjusted to pH 8.3 and 25 mL was dispensed onto each sheet. The containers were sealed and placed on an orbital shaker for 16 h at 22-24° C. After this time, the blocking solution was discarded, and sheets washed with DI water for 20 min. This was repeated once. Sheets were washed with 0.5 M AcOH for 20 min, followed by 0.1 M Tris, 0.15 M NaCl at pH 8.5. This two-step process was repeated once, followed by 2× DI water washes for 20 min each.

After blocking had been performed the material was fully immersed in 1:1:3 glycerol/ethanol/water and stored in the fridge for a minimum of 1 hour.

### Ligand Density Measurement:

Using a NanoDrop spectrophotometer, the AAV vector ligand concentration of each collected supernatant was calculated. This was used to calculate the mass of ligand immobilised. One disc was taken from each sheet where the supernatant was collected/concentration measured as described above. The thickness of each disc was taken at five points across the sheet, using a Mitutoyo Micrometer, to calculate an average thickness for the sheet. From this, the total volume of the sheet was calculated. The mass of ligand immobilised was divided by the volume of the sheet to calculate the ligand density. An average ligand density can be calculated for each batch.

With the above-described procedure, a ligand density of 3-7 mg/mL adsorbent was obtained, which for some ligands may correspond to approximately 0.5-5 μmol/g, e.g. 0.7-4.6 μmol/g). With such a ligand density it is possible to achieve static binding capacities in excess of 1E15 AAV5 capsids per mL of adsorbent, when AAV5 is used. The ligand density being measured using amino acid analysis or UV spectroscopic analysis of the pre-, post- and wash immobilisation solutions.

### Preparation of Polishing Fibro Prototype, Fibro-VS-DAX (NG):

This prototype comprises an anion ligand comprising N,N-diethylethylenediamine group (herein alternatively referred to as “DAX”), by use of an activation linker comprising vinyl sulfone (VS), not comprising glycidol (herein alternatively referred to as “non-glycidol” or “NG”).

Fibro-VS (NG) preparation: Fibro cellulose acetate (CA) sheets, prepared as described above for the affinity capture Fibro prototype, were inserted into a reactor with gauzes in between. The sheets were washed with MQ-water (3×5 L, 15 min per wash). The reactor was drained, the temperature increased to 30° C. and KOH reaction solution (309 g in 5500 mL 33% EtOH) added. The solution was circulated for 2 h. The reactor was drained and the sheets washed with MQ-water (4×5 L) and acetone (2×5 L). The reactor was drained and the sheets left to dry overnight. The reactor was then filled with 3 L sodium carbonate buffer (306.6 g Na₂CO₃ in 2960 mL MQ-water) and acetonitrile (1224 mL). The solution was cooled to 15° C. and then pumped to the reaction chamber. Divinyl sulfone (DVS) (945 mL) was added directly to the reaction chamber. The circulation was turned back on, and the reaction left to proceed at room temperature for 6 h. The reaction mixture was drained, and the sheets washed with 1:1 acetone/MQ-water (4×5 L) followed by MQ-water (4×5 L).

DAX coupling: The sheets were placed in food boxes and washed with MQ-water (4×100 mL, 20 min) on shaking table (84 rpm). To each box 25 mL MQ-water and 2 mL DAX were added. The boxes were placed on heated shaking tables (45° C., 75 rpm) for 23 h. The reaction solution was decanted, and the sheets were washed with MQ-water (6×100 mL, 20 min).

Deactivation: Thioglycerol (15.2 mL) was added to Tris buffer (600 mL, Tris 0.1 M, EDTA 0.001 M, pH 10) and the pH adjusted to 8.6. This solution (153 mL) was added to the food boxes and the reactions sealed and put on the shaking table (84 rpm) in room temperature for 16 h. The reaction solution was decanted, and the sheets were washed with 20% EtOH (3×100 mL, 20 min) and MQ-water (3×100 mL, 20 min). Titration gave an ionic capacity of 162 μmol/mL of membrane.

### Preparation of Chromatography Device:

Each of the Fibro chromatography material prototypes was assembled into a chromatography device of the type HiTrap Fibro™ (Cytiva, Sweden) 0.4 mL, herein alternatively called a Fibro unit.

### Results

The full and empty separation was similar for the in-line connected workflow and for a corresponding comparative step-by-step workflow with manual neutralisation, dilution, or buffer exchange (not shown).

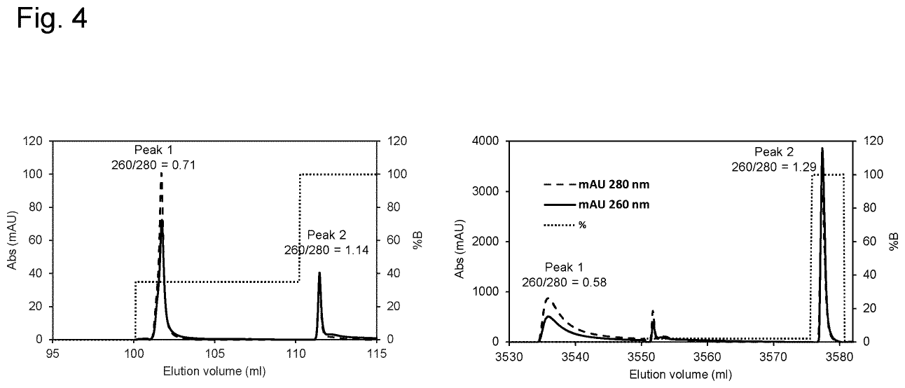

As shown in FIG. 4A and FIG. 4B, the anion exchange polishing chromatograms for the 1^{st} set-up and the 2^{nd} set-up showed similar separation results with the expected UV260:280 ratios for full capsids, i.e., Peak 2.

For the 1^{st} set-up, the empty capsids bound to the resin anion exchange polishing material and eluted at 33% of buffer B (Peak 1) while the full capsids bound and eluted at 100% of buffer B (Peak 2), see FIG. 4A.

For the 2^{nd} set-up, most of the empty capsids did not bind to the Fibro anion exchange polishing material but passed the polishing material in flow-through, hence the profile of Peak 1 is broader and flatter than for the 1^{st} set-up. Only a small amount of empty capsids were bound to the polishing material and eluted at 2% of buffer B. The full capsids bound and eluted at 100% of buffer B (Peak 2), see FIG. 4B.

### Example 2

Experimental designs for separation of fully packaged AAV5 capsids from empty AAV5 capsids are performed with equipment and samples as in Example 1 above, with the following variations:

In terms of first chromatography material (used in the capture step):
1) Ligand comprising a cation exchange group
2) Ligand comprising a multimodal group

In relation to both of the above-listed chromatography materials, one or more flow-through steps may be added between the capture step and the conditioning step. For any such additional flow-through step, a suitable chromatography material may comprise porous beads having an inner porous core and an outer porous shell, wherein the core is capable of binding molecules via hydrophobic interactions, and wherein the pore size of the shell prevents particles having a size of ≥20 nm from contacting the core.

### Example 3: Separation of Capsids of Different Adeno-Associated Virus Serotypes Under

Experimental designs for separation of full capsids from empty capsids of adeno-associated virus serotypes AAV1, AAV2, AAV4, AAV6, AAV7, AAV8, AAV10, AAV11, AAV12, and AAV13 are performed according to the variable conditions of Example 1 and Example 2 above.

### REFERENCES

WO2023285011 A1
U.S. Pat. No. 6,428,707
Xiaotong Fu et al, Analytical Strategies for Quantification of Adeno-Associated Virus Empty Capsids to Support Process Development, Human gene therapy methods, 2019, 30(4): 144-152
WO2018011599 A2
O. Hardick, et al, J. Mater. Sci. 46 (2011) 3890, Nanofibre fabrication in a temperature and humidity controlled environment for improved fibre consistency

1. A method for separating adeno-associated virus capsids from one or more impurities, said adeno-associated virus capsids being fully packaged with genetic material, said method comprising:
a. adding a feed comprising adeno-associated virus capsids fully packaged with genetic material, and one or more impurities, to a first chromatography device, comprising a first chromatography material comprising a support material functionalised with a ligand, wherein the ligand comprises an affinity group having a binding affinity for said adeno-associated virus capsids, an ion exchange group, or a multimodal group;
b. eluting said adeno-associated virus capsids from the first chromatography device in at least one eluate fraction;
c. adding the at least one eluate fraction comprising said adeno-associated virus capsids, obtained in step b, to a second chromatography device, comprising a second chromatography material comprising a conditioning chromatography material;
d. obtaining said adeno-associated virus capsids in at least one flow-through fraction from the second chromatography device;
e. adding the at least one flow-through fraction comprising said adeno-associated virus capsids, obtained in step d, to a third chromatography device, comprising a third chromatography material;
f. obtaining said adeno-associated virus capsids from the third chromatography device in at least one eluate fraction;
wherein the feed is continuously passed through the first, the second and the third chromatography devices, to enable separation of said adeno-associated virus capsids from one or more impurities, wherein the first, the second and the third chromatography devices are connected in series.

2. The method according to claim 1, wherein the method comprises applying a selection valve arrangement configured to enable said separation, by allowing the feed to continuously pass through the first, the second, and the third chromatography devices; wherein the selection valve arrangement comprises a first chromatography device selection valve.

3. The method according to claim 2, wherein the selection valve arrangement further comprises a second chromatography device selection valve.

4. The method according to claim 3, wherein the selection valve arrangement further comprises a third chromatography device selection valve.

5. The method according to claim 1, further comprising equilibrating the second chromatography material before step c to conditions required for the adeno-associated virus capsids to be obtained in step d, and to be obtained in step f.

6. The method according to claim 1, further comprising equilibrating the third chromatography material before step e to conditions required for the adeno-associated virus capsids to be obtained in step f.

7. The method according to claim 1, wherein the adeno-associated virus capsids are capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), adeno-associated virus serotype 10 (AAV10), adeno-associated virus serotype 11 (AAV11), adeno-associated virus serotype 12 (AAV12), or adeno-associated virus serotype 13 (AAV13), or a variant thereof.

8. The method according to claim 1, wherein the one or more impurities comprise adeno-associated virus capsids not fully packaged with genetic material.

9. The method according to claim 1, wherein the third chromatography material comprises a support material functionalised with a ligand, wherein the ligand comprises an anion exchange group or a multimodal group.

10. The method according to claim 9, wherein the ligand of the third chromatography material comprises an anion exchange group defined by Formula I: 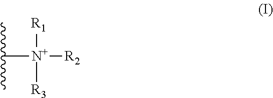
wherein R₁ is selected from H and C1-C3 alkyl, and R₂ and R₃ are independently selected from H, C1-C3 alkyl, CH2OH, and CH2CHOHCH3, preferably wherein each of R1, R₂, and R₃ is CH3.

11. The method according to claim 9, wherein the ligand of the third chromatography material comprises an anion exchange group defined by Formula II: 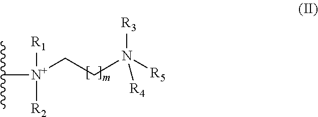
wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3;
provided that if m is 1, the ligand is defined by the following Formula III: 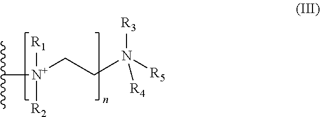
wherein n is an integer of from 0 to 3;
provided that if n is 0, R₃ and R₄ are independently selected from C1-C3 alkyl, and R₅ is hydrogen or CH2CHOHCH3.

12. The method according to claim 9, wherein the ligand of the third chromatography material comprises an anion exchange group defined by Formula IV: 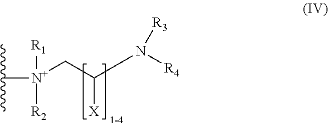
wherein X, for each occurrence independently, is selected from H, OH and a C₁₋₃ group, and R₁, R₂, R₃ and R₄ are independently selected from H and a C₁₋₃ group,
wherein a C₃ group is straight or branched,
wherein a C₁₋₃ group comprises groups independently selected from OH, O—C₁₋₂, S—C₁₋₂, NH, NHR, and NR₂,
wherein R is selected from H and a C₁₋₃ group.

13. The method according to claim 1, wherein the ligand of the first chromatography material comprises an affinity group which has a binding affinity for adeno-associated virus capsids.

14. The method according to claim 1, wherein the ligand of the first chromatography material comprises an anion exchange group defined by Formula I: 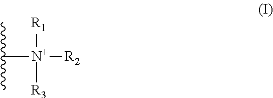
wherein R₁ is selected from H and C1-C3 alkyl, and R₂ and R₃ are independently selected from H, C1-C3 alkyl, CH2OH, and CH2CHOHCH3, preferably wherein each of R₁, R₂, and R₃ is CH3.

15. The chromatography system according to claim 1, wherein the ligand of the first chromatography material comprises a cation exchange group, wherein the cation exchange group is a sulfonate group.

16. The method according to claim 1, wherein the ligand of the first chromatography material comprises a multimodal group.

17. The method according to claim 16, wherein the multimodal group is a multimodal weak cation exchange group.

18. The method according to claim 16, wherein the multimodal group is a multimodal weak anion exchange group.

19. The method according to claim 1, wherein the support material of the first chromatography material comprises a membranous structure, nanofibres, a monolith, porous particles, non-porous particles, or expanded bed media.

20. The method according to claim 1, wherein the second chromatography material comprises a size exclusion chromatography material.

21. The method according to claim 1, wherein the support material of the third chromatography material comprises a membranous structure, nanofibres, a monolith, porous particles, non-porous particles, or expanded bed media.

22. A chromatography system, said system comprising:
a buffer valve arrangement configured to allow independent control of a first buffer feed and a second buffer feed;
a pump arrangement configured to supply a first buffer feed, a second buffer feed, and a feed comprising a biological target compound and one or more impurities;
a selection valve arrangement, comprising a first chromatography device selection valve;
a first chromatography device comprising a first chromatography material comprising a support material functionalised with a ligand, wherein the ligand comprises an affinity group having a binding affinity for a biological target compound, an ion exchange group, or a multimodal group;
a second chromatography device, comprising a second chromatography material, which comprises a conditioning chromatography material;
a third chromatography device comprising a third chromatography material;
wherein the selection valve arrangement is configured to enable separation of biological target compounds from impurities by allowing a feed, comprising biological target molecules and one or more impurities, to continuously pass through the first, the second, and the third chromatography devices, wherein the first, the second, and the third chromatography devices are configured to be connected in series.

23. The chromatography system according to claim 22, wherein the selection valve arrangement further comprises a second chromatography device selection valve.

24. The chromatography system according to claim 23, wherein the selection valve arrangement further comprises a third chromatography device selection valve.

25. The chromatography system according to claim 22, wherein the third chromatography material comprises a support material functionalised with a ligand, wherein the ligand comprises an anion exchange group or a multimodal group.

26. The chromatography system according to claim 25, wherein the ligand of the third chromatography material comprises an anion exchange group defined by Formula I: 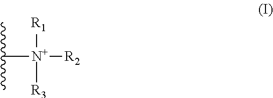
wherein R₁ is selected from H and C1-C3 alkyl, and R₂ and R₃ are independently selected from H, C1-C3 alkyl, CH2OH, and CH2CHOHCH3.

27. The chromatography system according to claim 25, wherein the ligand of the third chromatography material comprises an anion exchange group defined by Formula II: 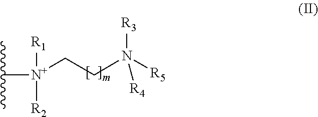
wherein:
m is an integer of from 1 to 3;
R₁ and R₂ are independently selected from a C1-C3 alkyl; R₃, and R₄ are independently selected from C1-C3 alkyl and CH2CHOHCH3; and R₅ is selected from hydrogen, a C1-C3 alkyl and CH2CHOHCH3;
provided that if m is 1, the ligand is defined by the following Formula III: 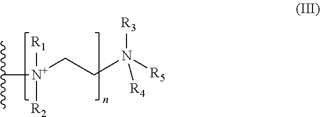
wherein n is an integer of from 0 to 3;
provided that if n is 0, R₃ and R₄ are independently selected from C1-C3 alkyl, and R₅ is hydrogen or CH2CHOHCH3.

28. The chromatography system according to claim 25, wherein the ligand of the third chromatography material comprises an anion exchange group defined by Formula IV: 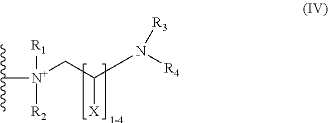
wherein X, for each occurrence independently, is selected from H, OH and a C₁₋₃ group, and R₁, R₂, R₃ and R₄ are independently selected from H and a C₁₋₃ group,
wherein a C₃ group is straight or branched,
wherein a C₁₋₃ group comprises groups independently selected from OH, O—C₁₋₂, S—C₁₋₂, NH, NHR, and NR₂,
wherein R is selected from H and a C₁₋₃ group.

29. The chromatography system according to claim 22, wherein the ligand of the first chromatography material comprises an affinity group which has a binding affinity for adeno-associated virus capsids.

30. The chromatography system according to claim 22, wherein the ligand of the first chromatography material comprises an anion exchange group defined by Formula I: 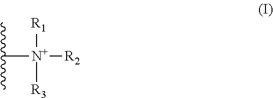
wherein R₁ is selected from H and C1-C3 alkyl, and R₂ and R₃ are independently selected from H, C1-C3 alkyl, CH2OH, and CH2CHOHCH3, preferably wherein each of R1, R₂, and R₃ is CH3.

31. The chromatography system according to claim 22, wherein the ligand of the first chromatography material comprises a cation exchange group, wherein the cation exchange group is a sulfonate group.

32. The chromatography system according to claim 22, wherein the ligand of the first chromatography material comprises a multimodal group, wherein the multimodal group is a multimodal weak cation exchange group or a multimodal weak anion exchange group.

33. The chromatography system according to claim 22, wherein the support material of the first chromatography material comprises a membranous structure, nanofibres, a monolith, porous particles, non-porous particles, or expanded bed media.

34. The chromatography system according to claim 22, wherein the second chromatography material comprises a size exclusion chromatography material.

35. The chromatography system according to claim 22, wherein the support material of the third chromatography material comprises a membranous structure, nanofibres, a monolith, porous particles, non-porous particles, or expanded bed media.

36. Use of a chromatography system according to claim 22, for separation of a biological target compound from one or more impurities, wherein the biological target compound is selected from adeno-associated virus capsids, such as capsids of adeno-associated virus serotype 1 (AAV1), adeno-associated virus serotype 2 (AAV2), adeno-associated virus serotype 3 (AAV3), adeno-associated virus serotype 4 (AAV4), adeno-associated virus serotype 5 (AAV5), adeno-associated virus serotype 6 (AAV6), adeno-associated virus serotype 7 (AAV7), adeno-associated virus serotype 8 (AAV8), adeno-associated virus serotype 9 (AAV9), adeno-associated virus serotype 10 (AAV10), adeno-associated virus serotype 11 (AAV11), adeno-associated virus serotype 12 (AAV12), or adeno-associated virus serotype 13 (AAV13), or a variant thereof.

37. The use according to claim 36, wherein the first chromatography material is selected from a group consisting of:
i. a support material in the form of porous particles functionalised with a ligand comprising an affinity group which has a binding affinity for adeno-associated virus capsids;
ii. a support material in the form of a convection-based membranous structure comprising a non-woven web of polymer nanofibres functionalised with a ligand comprising an affinity group which has a binding affinity for adeno-associated virus capsids;
iii. a support material in the form of porous particles functionalised with a ligand comprising a multimodal weak cation exchanger; and
iv. a support material in the form of porous particles functionalised with a ligand comprising a sulfonate group.

38. The use according to claim 36, wherein the third chromatography material is selected from a group consisting of:
i. a support material in the form of porous particles functionalised with a ligand defined by Formula I, wherein each of R1, R₂, and R₃ is CH3; wherein the ligand is connected to the support material by dextran; wherein the adeno-associated virus capsids are capsids of AAV2, AAV5, AAV8, or AAV9;
ii. a support material in the form of a convection-based membranous structure comprising a non-woven web of polymer nanofibres functionalised with a ligand defined by Formula I, wherein each of R1, R₂, and R₃ is CH3; wherein the adeno-associated virus capsids are capsids of AAV2, AAV5, or AAV8;
iii. a support material in the form of porous particles or a convection-based membranous structure comprising a non-woven web of polymer nanofibres, functionalised with a diethylethanolamine ligand; wherein the adeno-associated virus capsids are capsids of AAV2, AAV5, or AAV8; and
iv. a support material in the form of porous particles or a convection-based membranous structure comprising a non-woven web of polymer nanofibres, functionalised with a N,N-diethylethylenediamine ligand; wherein the adeno-associated virus capsids are capsids of AAV2, AAV5, or AAV8.
